(19) United States
(12) Patent Application Publication
(10) Pub. No.: US 2025/0325570 A1
(43) Pub. Date: Oct. 23, 2025

(54) DOSE AND REGIMEN FOR A HETEROCYCLIC PHOSPHINIC COMPOUND

(71) Applicant: PHOST'IN THERAPEUTICS, MONTPELLIER (FR)

(72) Inventors: Ludovic CLARION, COMPBAILLEUX (FR); Alain HERRERA, PARIS (FR); Karine CHORRO, CLAPIERS (FR); Séverine LOISEAU, VAILHAUQUES (FR); Carine JACQUARD, CAUSSE DE LA SELLE (FR)

(73) Assignee: PHOST'IN THERAPEUTICS, MONTPELLIER (FR)

(21) Appl. No.: 18/866,761

(22) PCT Filed: May 19, 2023
(86) PCT No.: PCT/EP2023/063459

§ 371 Date: Nov. 18, 2024

(30) Foreign Application Priority Data
May 20, 2022 (EP) 22305750.6

(51) Int. Cl. A61K 31/665

(52) U.S. Cl. CPC A61K 31/665

(57) ABSTRACT

The present invention relates to the use of heterocyclic phosphinic compounds or compositions comprising the same for treating cancer and/or for a use for reducing or preventing the appearance of metastases in a human patient afflicted with a cancer, wherein the compound is administered with a daily dose from 1 mg/kg to 80 mg/kg.

### TECHNICAL FIELD

The present invention relates to the use of a heterocyclic phosphinic compound or compositions comprising the same for treating cancer, wherein the compounds is administered by a specific dose regimen.

### BACKGROUND

In 2020, 2.7 million people across the European Union were diagnosed with cancer and 1.3 million people lost their lives to the disease, with it projected that mortality will increase by more than 24% by 2035 (EU Commission, 2021). The incidence of central nervous system (CNS) cancers globally was approximately 330,000 in 2016, with glioblastoma, also known as glioblastoma multiforme (GBM), being the most common form of primary CNS cancer (Patel et al., 2019). Among women, breast cancer is the main cancer site, expected to account for 29% of all new cancer cases (EU Commission, 2021) with 10-15% of breast carcinomas are known to be of the TNBC subtype (Dawood et al, 2012).

The mainstay of treatment options often includes a combination of different treatment modalities such as systemic therapies like chemotherapy alongside surgery and radiotherapy depending on the cancer origin, location and metastatic status. The introduction of monoclonal antibodies signaled an important change in the management of cancer, opening up a broader range of therapeutic targets whilst allowing a wider range of patients to be treated due to the more manageable side effect profiles of this class of agents comparative to traditional cytotoxic therapies. Newer therapeutic options have again shifted the treatment landscape significantly in the last 10 years with novel treatments becoming more widely available for numerous indications, including treatments harnessing the host immune system such as checkpoint inhibitors and advanced therapy medicinal products (ATMPs) like chimeric antigen receptor T cell therapy (CAR T).

Despite the variety of available therapies and advancements within them, there is a clear need for further options, especially in challenging to treat indications such as GBM and triple-negative breast cancer (TNBC) where there has been considerably less progress in mortality rates comparable to those in which the aforementioned treatments are effective. Some of the physiological characteristics of GBM contribute to the high mortality rate and treatment resistance associated with this condition, for example limited drug entry due to the blood-brain barrier and high efflux transporter presence causes reduced drug concentration within the tumour environment, and cellular heterogeneity making full control of the tumour mass challenging (Noch, Ramakrishna, & Magge, 2018). TNBC is characterised by higher rates of relapse and shorter overall survival comparative to other breast cancer types such as HER-2 or hormone receptor positive cancers (Garrido-Castro, Lin, & Polyak, 2019). Due to the lack of expression of major therapeutic targets commonly exploited in other breast cancer subtypes there is a paucity in effective treatment options available to TNBC patients which is reflected in their poorer prognosis, particularly in the metastatic setting (Huang et al., 2020).

The impact from the lack of development of new therapeutic options for harder to treat cancer types is reflected in recent mortality data as those cancers where therapy options have significantly advanced, such as melanoma and lung, have had significant reduction in mortality (6.2% and 4.2% respectively, p<0.05). Conversely GBM mortality has increased by 0.4% over the same period, with other difficult to treat cancer types showing a similar pattern (Henley et al., 2020).

This demonstrates an ongoing unmet need to develop a broader range of treatment options to close the widening gap in hard-to-treat cancers and deliver further treatment options to those who have exhausted all others.

GnT-V is a N-glycosylation enzyme that catalyses the transfer of N-acetylglucosamine (GlcNAc) to N-linked glycans, initiating the β1,6-branch of N-glycans (Kizuka & Taniguchi, 2016). The β1,6-branch is usually further elongated with alternating galactose and GlcNAc residues to form a polylactosamine structure that behaves as a high affinity ligand for galectins (Dennis, Nabi, & Demetriou, 2009) in addition to modifying protein conformation and consequent activities. Galectin-glycan interactions, which form the galectin lattice or glycocalyx, control membrane turnover of glycoproteins by increasing their retention time at the cell surface. GnT-V expression and activity has been found to be upregulated in various types of cancer, including in breast, colorectal, liver, gastric, oesophageal and brain cancers (Kizuka & Taniguchi, 2016) with very low expression seen in healthy tissues. In particular, glioma cells express high levels of GnT-V and consequently high β1,6-branched N-glycans, the product of GnT V activity (Yamamoto et al., 2000). Enhanced activity of GnT-V in tumour cells has been linked—through diverse mechanisms—to increased tumour cell proliferation, migration, invasiveness, resistance and immune escape, including in gliomas (Yamamoto et al., 2000) and in TNBC, where it has been seen that there are N-glycan polylactosamines associated with GnT-V distributed within tumour tissues (Scott et al., 2019).

There are currently no known anti-cancer drugs targeting GnT-V either in clinical use or under development.

### SUMMARY OF THE INVENTION

The Inventors have found that a family of heterocyclic phosphinic compounds, in particular compound 3-Hydroxy-4,5-bis-benzyloxy-6-benzyloxymethyl-2-phenyl-2-oxo-2,5-[1,2]oxaphosphinane, more particularly a crystalline polymorphic form of said compound, inhibits GnT-V activity. These compounds may thus be used as anti-cancer drugs targeting GnT-V, and for reducing or preventing the appearance of metastases in a patient afflicted with a cancer.

Besides, the inventors have found a specific dose regimen of these compounds, that ensures efficacy for treating cancer while reducing the risk of occurrence of adverse events.

The invention thus relates to a compound of general formula (1) as recited below, in particular to compound 3-Hydroxy-4,5-bis-benzyloxy-6-benzyloxymethyl-2-phenyl-2-oxo-2λ5-[1,2]oxaphosphinane, more particularly the crystalline polymorphic form of said compound, for use for treating cancer and/or reducing or preventing the appearance of metastases in a patient, preferably a human patient, afflicted with a cancer, wherein the compound is administered with a daily dose from 1 mg/kg to 80 mg/kg.

### BRIEF DESCRIPTION OF THE FIGURE

FIG. 1 illustrates a powder X-ray diffraction pattern for the crystalline form of the invention characterized by x-ray powder diffraction reflections at about 8.65, 16.06, 16.53, 19.16 and 21.05±0.20 degrees two-theta (as prepared in example 1 of WO 2018/054925).

### DETAILED DESCRIPTION OF THE INVENTION

The present invention relates to the use of heterocyclic phosphinic compounds of formula (1) as detailed below, and in particular compound 3-Hydroxy-4,5-bis-benzyloxy-6-benzyloxymethyl-2-phenyl-2-oxo-2λ5-[1,2]oxaphosphinane (also named as compound 3.1), with a specific dose regimen, for treating cancer while reducing the risk of occurrence of adverse events. Said compounds have been previously described as anti-cancer agents and in particular for reducing or preventing the appearance of metastases, as disclosed in PCT patent applications WO2009/004096 and WO2014/128429. Finally, the crystalline polymorphic form of 3-Hydroxy-4,5-bis-benzyloxy-6-benzyloxymethyl-2-phenyl-2-oxo-25-[1,2]oxaphosphinane has been disclosed in the international application WO 2018/054925.

The compounds for use according to the invention have the following formula (1):

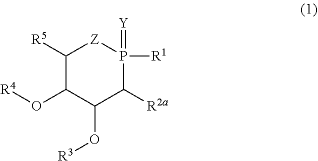

wherein Y represents an oxygen, a sulfur or a selenium atom, preferably an oxygen atom,
Z represents O, S, Se, NH or a NR₆ group, wherein R₆ is an aryl or an optionally substituted alkyl group, preferably an oxygen atom,
R¹ represents a hydrogen atom, an optionally substituted alkyl group or an aryl group,
R^{2a} represents a hydrogen atom, halogen, azide (N₃), a carbonate or dithiocarbonate group, a 1H-[1,2,3]triazolyl group or a group —X—R², wherein
X represents an oxygen, a sulfur, a selenium atom, a NH or NR₇ group, R₇ being an optionally substituted aryl or alkyl group; X preferably represents 0 or NH, and;
R2 represents an aryl group, an optionally substituted alkyl group, a hydrogen atom, a trichloroacetimidate group (—C(═NH)CCl₃), an acyl, formyl, sulfonyl, sulfinyl, tert-butyldiphenylsilyl, allyl group, a saccharyl, ester, amide, thioamide, sulfonamide group, or X—R₂ represents a P(O)R₂R₆ group, in which R₂ and R₆ represent independently from each other an aryl group, an optionally substituted alkyl group, OH, an alkoxy or an aryloxy group,
R³ and R⁴ represent independently from each other an aryl, an optionally substituted alkyl group, an hydrogen atom, a trichloroacetimidate group, an acyl, formyl, sulfonyl, sulfinyl, tert-butyldiphenylsilyl group, an allyl, a saccharyl, ester, amide, thioamide, sulfonamide group, or R³ and R⁴ taken together form a divalent radical of formula —R³—R⁴—, wherein —R³—R⁴— preferably represents an isopropylidene, benzylidene, diphenyl methylidene, cyclohexyl methylidene group, and their substituted analogues, for example a 4-methoxybenzylidene group, or a linear alkylene group such as an ethylene group (so as to form a propane-1,2-diol group),
R⁵ represents a hydrogen atom or a hydrocarbon group comprising one or more heteroatoms, preferably selected from oxygen, sulfur or nitrogen, more preferably oxygen.

In the present description of chemical compounds, the names are typically employed according to their usual definition.

As used herein, “alkyl” means a linear or branched, saturated or unsaturated hydrocarbon group, having from 1 to 25 carbon atoms, including in particular the acyclic groups with from 1 to 8 carbon atoms, such as methyl, ethyl, n-propyl, isopropyl, butyl, n-hexyl groups; cycloalkyl groups having preferably from 3 to 7 carbon atoms, cycloalkylmethyl groups having preferably from 4 to 8 carbon atoms.

As used herein, “substituted alkyl” means an alkyl group such as defined hereabove, that is bound through a sp3 carbon atom and substituted with one or more aryl groups and/or comprising one or more heteroatoms such as N, S or O. Suitable examples include arylalkyl groups such as (—CPh3)-trityl group, benzyl group (noted Bn) or 4-methoxybenzyl group, alkoxyalkyl groups, especially dialkoxymethyl groups such as diethoxymethyl or dimethoxymethyl groups, CH2CO2R11 groups, wherein R11 represents an optionally substituted alkyl group or an aryl group.

As used herein, “alkoxy” means an alkyl group that is bound to the rest of the molecule through an oxygen atom, for example an ethoxy, methoxy, or n-propoxy group.

As used herein, “aryloxy” means an aryl group bound to the rest of the molecule through an oxygen atom, for example a benzoxy group.

As used herein, “acyl” means a group derived from a carboxylic acid by removing the hydroxyl group, having preferably the formula —C(O)R8, wherein R8 represents an aryl or an optionally substituted alkyl group, for example an acetyl, trifluoro acetyl, propionyl, oleoyl, myristoyl or benzoyl group.

As used herein, “sulfonyl” means a group derived from a sulfonic acid by removing the hydroxyl group, having preferably the formula —SO2R9, wherein R9 represents an optionally substituted alkyl group or an aryl group.

As used herein, “sulfinyl” means a radical derived from a sulfinic acid by removing the hydroxyl group, having preferably the formula —SOR10, wherein R10 represents an optionally substituted alkyl group or an aryl group.

As used herein, “dithiocarbonate group” means a group of formula —OC(S)SR9c, wherein R9c represents an optionally substituted alkyl group or an aryl group.

As used herein, “carbonate group” means a group of formula —OC(O)OR9d, wherein R9d represents an optionally substituted alkyl group or an aryl group.

As used herein, an “ester group” means a group of formula —C(O)OR10′, wherein R10′ represents an optionally substituted alkyl group or an aryl group.

As used herein, an “amide group” means a group of formula —C(O)NR9′R9″, wherein R9′ represents an optionally substituted alkyl group or an aryl group and R9″ represents an optionally substituted alkyl group, an aryl group or a hydrogen atom.

As used herein, a “thioamide group” means a group of formula —C(S)NR9aR9b, wherein R9a represents an optionally substituted alkyl group or an aryl group and R9b represents an optionally substituted alkyl group, an aryl group or a hydrogen atom.

As used herein, a “sulfonamide group” means a group of formula —SO2NR11′R11″, wherein R11′ represents an optionally substituted alkyl group or an aryl group and R11″ represents an optionally substituted alkyl group, an aryl group or a hydrogen atom.

As used herein, “aryl” means an aromatic monovalent carbocyclic radical comprising only one ring (for example a phenyl group) or a plurality of fused rings (for example the naphthyl and terphenyl groups), which may optionally be substituted with one or more groups such as, without limitation, the alkyl (for example methyl), hydroxyalkyl, amino-alkyl, hydroxyl, thiol, amino, halogeno (fluoro, bromo, iodo, chloro), nitro, alkylthio, alkoxy (for example methoxy), aryloxy, mono-alkylamino, dialkylamino, acyl, carboxyl, alkoxycarbonyl, aryloxycarbonyl, hydroxysulfonyl, alkoxysulfonyl, aryloxysulfonyl, alkylsulfonyl, alkylsulfinyl, cyano, trifluoromethyl, tetrazolyl, carbamoyl, alkylcarbamoyl and dialkylcarbamoyl groups. Alternatively, two adjacent positions in the aromatic ring may be substituted with a methylenedioxy or ethylenedioxy group. As used herein, “aryl” also includes the “heteroaryl” groups, that is to say the aromatic rings wherein one or more carbon atoms of the one or more aromatic rings are substituted with one heteroatom such as a nitrogen, oxygen, phosphorus or sulfur atom. The heteroaryl groups may be one or several aromatic rings-containing structures or structures with only one or several aromatic rings coupled to one or more non aromatic rings. In structures possessing many rings, the rings may be fused, covalently bound or bound to each other through a divalent common group such as a methylene, ethylene or a carbonyl group. Suitable examples of heteroaryl groups include the thiophene groups (2-thienyl, 3-thienyl), pyridine groups (2-pyridyl, 3-pyridyl, 4-pyridyl), isoxazole, phthalimide, pyrazole, indole and furan groups, as well as their benzofused analogues, phenyl pyridyl ketone, quinoline, phenothiazine, carbazole and benzopyranone.

As used herein, a “saccharyl group” includes all radicals derived by removing a hydroxyl group or a hydrogen atom (preferably a hydroxyl group), from a natural or synthetic, protected or unprotected carbohydrate or sugar. The saccharyl group can include the monosaccharyl or oligosaccharyl groups, such as disaccharyl groups. The saccharyl groups, for example glucosyl and mannosyl groups may be derived from sugars such as, without limitation, the glucuronic acid, the lactose, the sucrose, the maltose, the allose, the alltrose, the glucose, the mannose, the idose, the galactose, the talose, the ribose, the arabinose, the xylose, the lyxose, the fructose, the threose, the erythrose, the [beta]-D-N-acetylgalactosamine, the [beta]-D-N-acetylglucosamine, the fucose, the sialic acid, the N-acetylneuraminic acid, the N-acetylmuramic acid, the glucosamine, the galactosamine, the rhamnose and their protected or substituted analogues, that are substituted for example with acyl, alkyl, aryl, halogeno and amino groups, as well as their desoxy type analogues.

As used herein, an oligosaccharyl group means a saccharyl group derived from at least two covalently bound monosaccharides, comprising preferably from 1 to 3 saccharide units. For a description of saccharide type structures, see “Essentials of Glycobiology,” Varki and al. Eds., Chapter 2 (Cold Spring Harbor Press, Cold Spring Harbor, N.Y., 1999). Preferred saccharyl groups are monosaccharyl groups. In compounds of formula (1), when R^{2a} represents —X—R2 group, wherein R2 represents a saccharyl group, said saccharyl group is preferably bound through a X group representing 0 or NH, preferably 0.

As used herein, a “saccharide” means a monosaccharide or an oligosaccharide.

“Bn” stands for a benzyl group, “Ac” an acetyl group.

Some compounds of the invention may equally present in a solvated or a non-solvated form, for example as an hydrate. Generally, solvated forms are equivalent to non-solvated forms and are included within the frame of the invention. Some compounds of the invention may have a plurality of various crystalline or amorphous forms. Generally, all physical forms are equivalent for the uses that are intended according to the present invention and are included within the frame of the present invention.

The compounds of the invention have several asymmetric (optical) centers, so that enantiomers or diasteroisomers may exist. It is understood that the present invention does include all the enantiomers and diasteroisomers of the compounds of formula (1), as well as their mixtures, especially those based on racemates. The different isomers may be separated according to methods known to those skilled in the art, notably silica gel chromatography- or fractional crystallisation-based methods.

The preferred compounds of formula (1) are those wherein Y=Z=O, that is to say 1,2-oxaphosphinane 2-oxide compounds.

In the compounds of the invention, R1 substituent, where it does not represent a hydrogen atom, is always bound to the intracyclic phosphorus atom through a carbon atom.

Preferred R¹ groups include H, alkyl groups, such as 2-benzyloxyethyl, ethyl, n-butyl, 3-phenylpropyl, n-octyl, dialkoxymethyl groups such as a diethoxymethyl or dimethoxymethyl group, aryl groups, such as phenyl, 4-methylphenyl, 4-nitrophenyl, 4-aminophenyl, 4-methoxyphenyl, 3,4-difluorophenyl, 2-thienyl, 4-fluorophenyl, 4-biphenyl, 3-methylphenyl, 3-methoxyphenyl and 3,5-difluorophenyl groups, as well as the following groups:

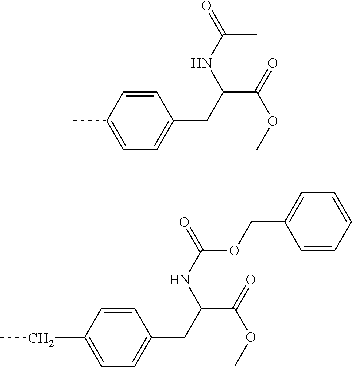

In a particular embodiment, R¹ is a phenyl group.

Preferred R2 groups include H, arylsulfonyl, methylsulfonyl, trichloroacetimidate, benzyl, saccharyl and aryl groups, such as phenyl, 4-methylphenyl, 4-nitrophenyl, 4-aminophenyl, 3,4-difluorophenyl, 3,5-difluorophenyl, and 3,4-dinitrophenyl groups.

Preferred X—R2 groups include O-aryl, OH, NH2, NH-aryl, S-aryl and dithiocarbonate groups, or NHCH2CO2R11, wherein R11 is such as defined hereabove, NHC(O)R12, wherein R12 represents an aryl group or an optionally substituted alkyl group, O—SO2R9 wherein R9 is such as defined hereabove, NH-Bn, O-saccharyl, OC(═NH)CCl3, phosphonic acid, phosphinic acid or phosphine oxide, urea, thiourea, carbamate and carbonate groups.
According to a particular embodiment, X—R2 is OH, and preferably R1 is a phenyl group.

Preferably, R³ and R⁴ represent independently from each other, a hydrogen atom, a benzyl, benzoyl or an acetyl group, or they form together a divalent radical of formula —R³—R⁴— representing preferably an isopropylidene group.

According to a particular embodiment, R³ and R⁴ represent a benzyl group and/or R¹ is a phenyl group and/or X—R² is OH.

According to another particular embodiment, R³ and R⁴ represent a benzyl group and preferably R¹ is a phenyl group and/or X—R² is OH.

According to a preferred embodiment of the invention, R⁵ is such that the compounds of formula (1) have the following formula (2) or (3):

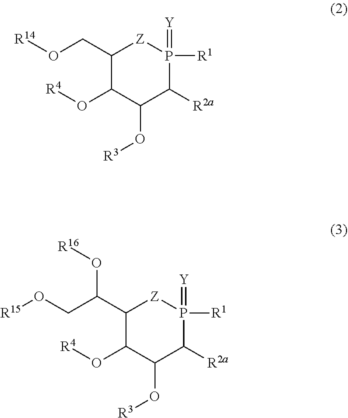

wherein R¹, R^{2a}, R³, R⁴, Y and Z are as defined hereabove, R¹⁴, R¹⁵ and R¹⁶ represent, independently from each other, a hydrogen atom, an aryl, an optionally substituted alkyl group, a trichloroacetimidate group, an acyl, formyl, sulfonyl, sulfinyl, tert-butyldiphenylsilyl group, an allyl, ester, amide, thioamide, sulfonamide group, or R¹⁵ and R¹⁶, taken together, form a divalent radical of formula —R¹⁵—R¹⁶—, wherein —R¹⁵—R¹⁶— preferably represents an isopropylidene, benzylidene, diphenyl methylidene, cyclohexyl methylidene group, and their substituted analogues, for example a 4-methoxybenzylidene group, or a linear alkylene group such as an ethylene group.

According to a particular embodiment, R¹⁴ represents a benzyl group, and preferably with at least one or more particular embodiments as above detailed, including where R³ and R⁴ represent a benzyl group and/or R¹ is a phenyl group and/or X—R2 is OH.

R⁵ when not representing a hydrogen atom, does preferably have from 1 to 25 carbon atoms, preferably from 1 to 20 carbon atoms, more preferably from 1 to 10 carbon atoms, and even more preferably from 1 to 8 carbon atoms. R may represent an optionally substituted alkyl group comprising one or more heteroatoms preferably selected from oxygen, sulfur or nitrogen, more preferably oxygen. Preferred R groups include alkoxyalkyl groups such as benzyloxymethyl (—CH₂OBn), —CH₂OH, 2,2-dimethyl-[1,3]-dioxolan-4-yl and 1,2-dihydroxy-ethyl CH(OH)CH₂OH groups, which means in the formulas (2) and (3) that R¹⁴=H or Bn, and R¹⁵=R¹⁶=H or R¹⁵ and R¹⁶, taken together, do form an isopropylidene radical.

According to a particular embodiment, the compound for use according to the invention is selected in the group consisting of
3-Hydroxy-4,5-bis-benzyloxy-6-benzyloxymethyl-2-phenyl-2-oxo-2λ5-[1,2]oxaphosphinane,
4-(2,2-Dimethyl-[1,3]dioxolan-4-yl)-2,2-dimethyl-2-oxo-2-phenyl-tetrahydro-6 λ*5*-[1,3]dioxolo[4,5-d][1,2]oxaphosphinan-3-aminobenzyl, more specifically (3aR,6S,7S,7aS)-7-(benzylamino)-4-((R)-2,2-dimethyl-1,3-dioxolan-4-yl)-2,2-dimethyl-6-phenyltetrahydro-[1,3]dioxolo[4,5-d][1,2]oxaphosphinine 6-oxide,
N-((2S,3S,4S,5S,6R)-4,5-bis(benzyloxy)-6-((benzyloxy)methyl)-2-oxido-2-phenyl-1,2-oxaphosphinan-3-yl)acetamide,
4,5-bis-benzyloxy-6-benzyloxymethyl-phenyl-2-oxo-2λ5-[1,2]oxaphosphinan-3-aminobenzyl,
more specifically (2S,3S,4S,5S,6R)-3-(benzylamino)-4,5-bis(benzyloxy)-6-((benzyloxy)methyl)-2-phenyl-1,2-oxaphosphinane,
(2S,3S,4S,5S,6R)-4,5-bis(benzyloxy)-6-((benzyloxy)methyl)-3-hydroxy-2-(4-phenoxyphenyl)-1,2-oxaphosphinane 2-oxide, and
(3aR,6R,7R,7aS)-4-((R)-2,2-dimethyl-1,3-dioxolan-4-yl)-2,2-dimethyl-6-oxido-6-phenyltetrahydro-[1,3]dioxolo[4,5-d][1,2]oxaphosphinin-7-yl benzoate.

In a more particular embodiment, the compound for use according to the invention is 3-Hydroxy-4,5-bis-benzyloxy-6-benzyloxymethyl-2-phenyl-2-oxo-2λ5-[1,2]oxaphosphinane.

Preparation of compound 3-Hydroxy-4,5-bis-benzyloxy-6-benzyloxymethyl-2-phenyl-2-oxo-2λ5-[1,2]oxaphosphinane can for example be carried out as described in WO 2009/004096, WO 2014/128429, and WO 2018/054925.

The compound 3-Hydroxy-4,5-bis-benzyloxy-6-benzyloxymethyl-2-phenyl-2-oxo-2,5-[1,2]oxaphosphinane to be used according to the invention has preferably the following Formula (I):

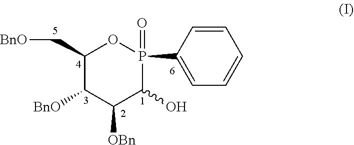 [SMILES: O=[P@@]1(c2ccccc2)O[C@H](COCc2ccccc2)[C@@H](OCc2ccccc2)[C@H](OCc2ccccc2)C1O]

Accordingly, the invention relates to a compound of formula (1), preferably 3-Hydroxy-4,5-bis-benzyloxy-6-benzyloxymethyl-2-phenyl-2-oxo-2λ5-[1,2]oxaphosphinane, and more preferably of formula (I), also called PST3.1, for use for treating cancer and/or reducing or preventing the appearance of metastases in a patient afflicted with a cancer, wherein the compound is administered with a daily dose from 1 mg/kg to 80 mg/kg.

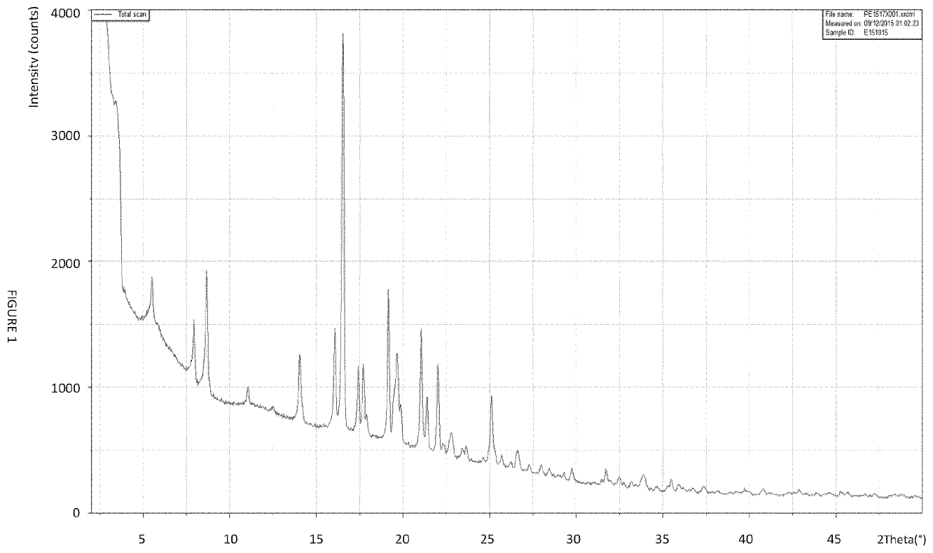

In a preferred aspect, the compound for use according to the invention is a compound of formula (I) in a crystalline form, characterized by powder x-ray diffraction reflections at about 8.65, 16.06, 16.53, 19.16 and 21.05±0.2, preferably ±0.1, degrees two-theta, this crystalline form can be further characterized by powder x-ray diffraction reflections at about 14.04, 17.69, 19.66, 22.02 and 25.12±0.2 degrees two-theta, or substantially as depicted in FIG. 1 or Table 1 below.

TABLE 1

| 2θ | D_{spacing} | Intensity | Intensity |
|---|---|---|---|
| ° | Å | counts | % |
| 2.35 | 37.56 | 585 | 2 |
| 5.50 | 16.05 | 4319 | 14 |
| 7.92 | 11.16 | 4759 | 15 |
| 8.65 | 10.21 | 9662 | 31 |
| 11.07 | 7.99 | 1455 | 5 |
| 12.54 | 7.05 | 594 | 2 |
| 14.04 | 6.30 | 5352 | 17 |
| 16.06 | 5.51 | 7852 | 25 |
| 16.53 | 5.36 | 31404 | 100 |
| 17.40 | 5.09 | 5099 | 16 |
| 17.69 | 5.01 | 5410 | 17 |
| 17.90 | 4.95 | 1486 | 5 |
| 19.16 | 4.63 | 11914 | 38 |
| 19.66 | 4.51 | 7009 | 22 |
| 19.84 | 4.47 | 2982 | 9 |
| 21.05 | 4.22 | 9412 | 30 |
| 21.36 | 4.16 | 4149 | 13 |
| 22.02 | 4.03 | 6997 | 22 |
| 22.31 | 3.98 | 782 | 2 |
| 22.78 | 3.90 | 1851 | 6 |
| 23.44 | 3.79 | 882 | 3 |
| 23.65 | 3.76 | 1083 | 3 |
| 24.62 | 3.61 | 481 | 2 |
| 25.12 | 3.54 | 5446 | 17 |
| 25.70 | 3.46 | 974 | 3 |
| 26.25 | 3.39 | 561 | 2 |
| 26.59 | 3.35 | 1515 | 5 |
| 27.27 | 3.27 | 603 | 2 |
| 27.98 | 3.19 | 798 | 3 |
| 28.45 | 3.13 | 630 | 2 |
| 29.29 | 3.05 | 557 | 2 |
| 29.74 | 3.00 | 1040 | 3 |
| 31.47 | 2.84 | 328 | 1 |
| 31.71 | 2.82 | 1157 | 4 |
| 31.95 | 2.80 | 336 | 1 |
| 32.50 | 2.75 | 623 | 2 |
| 33.18 | 2.70 | 401 | 1 |
| 33.86 | 2.64 | 1061 | 3 |
| 35.31 | 2.54 | 343 | 1 |
| 35.47 | 2.53 | 866 | 3 |
| 35.91 | 2.50 | 498 | 2 |
| 37.36 | 2.41 | 472 | 2 |
| 42.87 | 2.11 | 418 | 1 |
| 45.26 | 2.00 | 318 | 1 |

Preferably, the crystalline form of compound I for use according to the invention has less than about 20% of any other form of compound I present, more preferably has less than about 10% of any other form of compound I present, even more preferably is in a substantially pure form, i.e. has less than about 5% of any other compound I form present, and most preferably has less than about 2% of any other compound I form present.

In a particular embodiment, the crystalline form of compound I of the invention has a melting point, by Differential Scanning Calorimetry (DSC), of 175.5-177.5° C., more specifically 176.2° C. 0.4° C. (or ±0.3° C.), at a heating rate of 10° C./min.

In a particular embodiment, this crystalline form of compound I shows no significant weight loss, measured by thermal gravimetric analysis (“TGA”) at the range of about 25° C. to 250° C., i.e. before and after its melting point. In a particular embodiment, water content of up to about 0.3% (w/w) was measured by Karl Fisher.

In a particular embodiment, the crystalline form of the invention is non-hygroscopic. More specifically, the Dynamic vapor sorption (DVS) analysis on the crystalline form of the invention shows weight loss lower than 0.1% on the relative humidity range studied (0% RH to 95% RH).

According to a particular embodiment, the particle size measured by laser diffraction methods vary as follow: D10: from 70-80 nm, and/or D50: from 140-160 nm, and/or D90: 360-380 nm. D10, D50, and D90 represent respectively the mean particle size of 10%, 50%, and 90% of the number of the smallest particles measured by laser diffraction methods. For example, the D10 particle size is the size at which 10% of the particles is comprised of smaller particles, and the D50 is the size at which 50% of particles is comprised of smaller particles.

The method for preparing the crystalline form of compound I for use according to the invention is disclosed in WO 2018/054925.

According to the invention, a compound as defined herein is for use for treating cancer and/or reducing or preventing the appearance of metastases in a patient afflicted with a cancer, wherein the compound is administered with a daily dose from 1 mg/kg to 80 mg/kg.

The present invention further provides for a use of a compound of general formula (1) as defined herein, in particular a compound of formula (I) as defined herein, more particularly the crystalline polymorphic form of said compound as defined herein, for the manufacture of a medicament or pharmaceutical composition for treating cancer and/or for reducing or preventing the appearance of metastases in a patient afflicted with a cancer, wherein the compound is administered with a daily dose from 1 mg/kg to 80 mg/kg.

The present invention further provides for a method for treating cancer and/or for reducing or preventing the appearance of metastases in a patient afflicted with a cancer by administering in a patient in need of such treatment a daily dose from 1 mg/kg to 80 mg/kg of a compound of general formula (1) as defined herein, in particular a compound of formula (I) as defined herein, more particularly the crystalline polymorphic form of said compound as defined herein.

In particular, a compound of general formula (1) as defined herein, in particular the compound of formula (I) as defined herein, more particularly the crystalline polymorphic form of said compound as defined herein, when administered with a daily dose from 1 mg/kg to 80 mg/kg, is useful as an active principle in pharmaceutical compositions for human or veterinary use, preferably human use, intended for treating cancer (metastatic or primary), i.e. cancer cells, or for preventing the appearance of cancer, especially for reducing or preventing the appearance of metastases in a patient afflicted by a cancer. In the case where the patient is afflicted by a metastatic cancer, a compound as described herein, when administered with a daily dose from 1 mg/kg to 80 mg/kg, is especially directed in particular toward reducing or preventing the appearance of additional metastases.

In the present description, a patient denotes both an animal, in particular a non-human mammal, and a human. Preferably, the patient is a human. The term “patient afflicted by a cancer” means both a patient afflicted by a declared cancer (primary or metastatic) and a hidden cancer, i.e. invisible, the existence of which has been revealed, for example, by the discovery of metastases.

According to a particular embodiment, the patient is with solid tumour(s), in particular the patient is with advanced and/or metastatic solid tumour(s).

According to a particular embodiment, the patient may have received a previous line of treatment and/or the patient may be not or may be no longer responsive to other treatments.

In the present invention, cancer cells denote cells having typical characteristics of cells that cause cancer, such as uncontrolled proliferation, immortality, metastatic potential, rapid growth and high speed of proliferation, and certain specific morphological characteristics. Cancer cells are often in the form of a tumor, but such cells may exist alone in the body, or may be non-tumor-forming cancer cells, such as leukemic cells. Cancer cells may be associated with numerous types of cancers, comprising, without limitation, leukemia, a lymphoma, a melanoma, a neuroblastoma, liver cancer, ovarian cancer, brain cancer, lung cancer, bowel cancer, breast cancer, pancreatic cancer, prostate cancer, testicular cancer, esophageal cancer, uterine cancer, cervical cancer, kidney cancer, stomach cancer, bladder cancer, a cerebrospinal cancer or a colorectal cancer.

A compound as defined herein when administered with a daily dose from 1 mg/kg to 80 mg/kg, may be used for the therapeutic treatment of at least one of the cancers mentioned above.

When a compound as defined herein is used in the context of an antimetastatic treatment, the patient is afflicted with a “primary” cancer. This cancer is a cancer that is capable of metastatizing, which may be, without limitation, a melanoma, a glioblastoma multiform, a lung cancer, especially non-small-cell lung cancer, bowel cancer or colorectal cancer, breast cancer, prostate cancer, testicular cancer, cervical cancer, kidney cancer, preferably a glioblastoma multiform, breast cancer or non-small-cell lung cancer. The compounds as disclosed herein are particularly suited for treating the risk of metastasis in a patient afflicted with a glioblastoma multiform. It is now recognized that glioblastoma multiform (GBM), commonly known as glioblastoma, may be a cancer with metastatic potential giving rise to a generalized pathology (Schonsteiner, S. S. et al., Journal of Clinical Oncology 2011, 29, 23, 668-671). Cancer cells originating from glioblastomas may effectively cross the blood-brain barrier and establish extraneural metastases. The reported sites of extraneural metastases are the lungs, the pleura, the liver, cervical lymphatic nodules, bones and bone marrow.

In a preferred aspect of the invention, the cancer is in particular a solid tumour cancer, more particularly selected from non-small-cell lung carcinoma (NSCLC), small-cell lung carcinoma (SCLC), breast cancer, oesophageal cancer, melanoma, gastric cancer, GBM, small bowel cancer, colorectal cancer, anal cancer, pancreatic cancer, ovarian cancer, uterine cancer, cervical cancer, preferably from glioblastoma multiform, breast cancer and non-small-cell lung cancer, more preferably glioblastoma multiform

According to the invention, a compound as defined herein is administered with a daily dose from 1 mg/kg to 80 mg/kg, advantageously with a daily dose from 2 mg to 70 mg/kg. In particular, the daily dose to be administered to the subject may be from 2 mg/kg to 60 mg/kg, preferably from 2 mg/kg to 50 mg/kg, more preferably from 2 mg/kg to 40 mg/kg, more preferably from 2 mg/kg to 30 mg/kg, more preferably from 2 mg/kg to 20 mg/kg. In another specific embodiment, the daily dose to be administered to the subject may be from 5 mg/kg to 80 mg/kg, advantageously from 5 mg to 70 mg/kg, in particular from 5 mg/kg to 60 mg/kg, preferably from 5 mg/kg to 50 mg/kg, more preferably from 5 mg/kg to 40 mg/kg, more preferably from 5 mg/kg to 30 mg/kg, more preferably from 5 mg/kg to 20 mg/kg. In another specific embodiment, the daily dose to be administered to the subject may be from 10 mg/kg to 80 mg/kg, advantageously from 10 mg to 70 mg/kg, in particular from 10 mg/kg to 60 mg/kg, preferably from 10 mg/kg to 50 mg/kg, more preferably from 10 mg/kg to 40 mg/kg, more preferably from 10 mg/kg to 30 mg/kg, more preferably from 10 mg/kg to 20 mg/kg. More particularly, the daily dose to be administered to the subject may be about 2 mg/kg, 5 mg/kg, 10 mg/kg, 15 mg/kg 20 mg/kg, 25 mg/kg, 30 mg/kg, 35 mg/kg, 40 mg/kg, 45 mg/kg 50 mg/kg, 55 mg/kg 60 mg/kg, 65 mg/kg, 70 mg/kg, 75 mg/kg or 80 mg/kg. The daily dose being expressed in mg/kg body weight of the subject.

The compound of the invention can be administered once or several times a day. Advantageously, a compound as defined herein is administered from 2 to 6 times per 24 hours, in particular 2 to 5 times per 24 hours, in particular from 2 to 4 times per 24 hours, more particularly 2 or 3 times per 24 hours, advantageously twice per 24 hours, to reach the daily dose.

The duration of treatment with the compound of the invention may last as long as the symptoms of the disease persists. For example, the compound of the invention may be administered in 21-day cycles until the symptoms of the diseases disappear. Alternatively, and in particular in the context of the prevention of the appearance of metastases, the duration of treatment with the compound of the invention may be indefinite.

Advantageously, a compound as defined herein may be administered via oral, topical, parenteral, nasal, intravenous, percutaneous, transcutaneous, rectal, perlingual or airway administration. Preferably, the daily dose from 1 mg/kg to 80 mg/kg of a compound as defined herein, is administered orally.

In a particular embodiment of the invention, a compound as defined herein is administered orally, 2 or 3 times, preferably 2 times, per 24 hours, in order to reach the daily dose from 1 mg/kg to 80 mg/kg.

In a further embodiment, a compound as defined herein is administered orally, 2 or 3 times, preferably 2 times, per 24 hours, in order to reach the daily dose from 2 mg to 70 mg/kg, in particular from 2 mg/kg to 60 mg/kg, preferably from 2 mg/kg to 50 mg/kg, more preferably from 2 mg/kg to 40 mg/kg, more preferably from 2 mg/kg to 30 mg/kg, more preferably from 2 mg/kg to 20 mg/kg.

In another specific embodiment, a compound as defined herein is administered orally, 2 or 3 times, preferably 2 times, per 24 hours, in order to reach the daily dose from 5 mg/kg to 80 mg/kg, advantageously from 5 mg to 70 mg/kg, in particular from 5 mg/kg to 60 mg/kg, preferably from 5 mg/kg to 50 mg/kg, more preferably from 5 mg/kg to 40 mg/kg, more preferably from 5 mg/kg to 30 mg/kg, more preferably from 5 mg/kg to 20 mg/kg.

In another specific embodiment, a compound as defined herein is administered orally, 2 or 3 times, preferably 2 times, per 24 hours, in order to reach the daily dose from 10 mg/kg to 80 mg/kg, advantageously from 10 mg to 70 mg/kg, in particular from 10 mg/kg to 60 mg/kg, preferably from 10 mg/kg to 50 mg/kg, more preferably from 10 mg/kg to 40 mg/kg, more preferably from 10 mg/kg to 30 mg/kg, more preferably from 10 mg/kg to 20 mg/kg.

Advantageously, a compound as defined herein is administered in a fasted or fed subject, in particular in a fed subject. In the context of the invention, fasted conditions are defined as no food intake for 2 hours before and 2 hours after the compound administration, and fed conditions are defined as having received a regular meal within 1 hour, preferably within 30 minutes, before the compound administration.

A compound of general formula (1) as defined herein, in particular the compound of formula (I) as defined herein, more particularly the crystalline polymorphic form of said compound as defined herein, may be provided in a pharmaceutical composition for use for treating cancer and/or reducing or preventing the appearance of metastases in a patient afflicted with a cancer, wherein the compound is administered with a daily dose from 1 mg/kg to 80 mg/kg. The pharmaceutical composition may additionally comprise a pharmaceutically acceptable excipient, adjuvant and/or carrier.

The pharmaceutical composition of the invention may be in a solid or a liquid form. In a liquid form, the pharmaceutical composition preferably comes as an aqueous suspension or as a non-aqueous suspension, or as a water-in-oil or an oil-in-water emulsion.

The pharmaceutical compositions of the invention are typically suitable for the oral, topical, parenteral, nasal, intravenous, percutaneous, transcutaneous, rectal, perlingual or airway administration, preferably for oral administration.

To be administered by the oral route, a pharmaceutical composition according to the invention may present in the form of tablets, capsules, coated tablets, syrups, suspensions, solutions, powders, pellets, emulsions, suspensions of microspheres or nanospheres, lipid vesicle suspensions or various polymer-based vesicles.

To be administered by the oral route, a pharmaceutical composition according to the invention may be in the form of tablets that may be obtained from solid compositions comprising various excipients such as microcrystalline cellulose, sodium citrate, calcium carbonate, dicalcium phosphate or glycine. Various disintegrating agents such as starch (corn, potato or tapioca starch, etc.), alginic acid or a silicate may be used. Binders such as polyvinyl pyrrolidone, sucrose, gelatin, or acacia may also be used. Lubricants such as magnesium stearate, sodium laurylsulfate, or even talc may also be used. Such solid compositions, as a powder, may be used for preparing gelatin capsules. For solid compositions, lactose or polyethylene glycol with a high molecular weight may also be used.

To be administered by the oral route, a pharmaceutical composition according to the invention may also be in the form of liquid compositions. In this aspect, the composition may comprise various sweeteners, flavouring agents, colouring agents, possibly together with emulsifying agents or suspending agents, in combination with diluents such as water, ethanol, propylene glycol, glycerin or any combination of these excipients.

Generally speaking, a pharmaceutical composition according to the invention comprises from 0.01% to 99% by weight, and advantageously from 1% to 90% by weight, of a compound as defined herein, as compared to the total weight of the composition, so that the daily dose required can be administered in the subject within the required number of intakes.

Generally speaking, a pharmaceutical composition according to the invention comprises from 1% to 99.99% by weight, and advantageously from 10% to 99% by weight of an excipient or a mixture of pharmaceutically acceptable excipients.

Further pharmaceutical compositions that can be used according to the invention are as disclosed in WO 2018/054925.

The invention is further described with reference to the following, non-limiting, examples.

### EXAMPLES

### Example 1—Clinical trial: administration of the crystalline form of 3-Hydroxy-4,5-bis-benzyloxy-6-benzyloxymethyl-2-phenyl-2-oxo-2λ5-[1,2]oxaphosphinane, also called PST3.1a (=crystalline form of compound of formula (I) as described above)

### Clinical Background Information

The proposed clinical trial is a first-in-human trial (FIH) in patients with advanced solid tumours, including a dose escalation phase (part I) enrolling patients with any type of tumour with safety/tolerability as the primary endpoint, pharmacokinetics, evaluation of a potential food effect, and efficacy as secondary endpoints, followed by an expansion phase (part II) recruiting patients with glioblastoma (GBM—23 patients, cohort 1), triple negative breast cancer (TNBC—15 patients, cohort 2) and other selected solid tumours (30 patients, cohort 3). The dose for the expansion phase is selected based on the results of the escalation phase. Pharmacodynamics and response prediction is an exploratory objective of this whole FIH study.

The study enrols adult patients with advanced solid malignancies that are metastatic or unresectable with documented progression on a previous line of treatment and for which no further standard of care options are available.

### Dose Escalation Phase

In the dose escalation phase, patients with any solid tumour type may be included, but preferably patients affected by rNSCLC, SCLC, breast cancer, oesophageal cancer, melanoma, gastric cancer, GBM, small bowel cancer, colorectal cancer, anal cancer, pancreatic cancer, ovarian cancer, uterine cancer, cervical cancer, hepatocarcinoma).

### Objectives and Endpoints

### Primary Objective and Endpoint:

To determine the safety and tolerability of PST3.1a in patients with advanced solid tumours in a dose escalation procedure by determining the Maximal Tolerated Dose (MTD).

### Secondary Objectives and Endpoints:

To characterize the pharmacokinetics (PK) profile of PST3.1a by establishing the PK profile of the drug including assessment of a potential food effect on drug exposure.

To evaluate the efficacy of PST3.1a by determining the objective response rate (ORR), progression-free survival (PFS), and overall survival (OS).

### Exploratory Objective:

To characterize the pharmacodynamic effect of PST3.1a by determining changes in immune infiltration, glycolytic activity, membrane glycosylation, miRNAs expressed and circulating glycans in tumour and fluid samples before and after treatment (PD markers).

To identify predictive response biomarkers by profiling metabolic and mesenchymal responsive patterns.

### Dose Escalation

A standard 3+3 design is followed for the escalation phase. PST3.1a is administered orally twice a day (bid) continuously in cycles of 21 days. At each Dose Level (DL), three patients are included and the first patient is observed for at least 21 days before enrolling the following two. Three additional patients are enrolled at each DL if a Dose Limiting Toxicity (DLT) is observed in the first three patients. No intra-patient dose escalation is allowed. The Maximum Tolerated Dose (MTD) is defined as the highest dose at which a DLT is observed in 0/3 or 1/6 patients and is considered as the recommended phase 2 dose (RP2D).

The dose escalation phase includes two steps:
In step 1, one dose (DL1) of PST3.1a is tested, corresponding to the dose of 10 mg/kg/day bid.

Two full-course PK profiling are obtained for each patient: one at C1D1 (Cycle 1/Day 1) in fasted state patients and another at C2D1 (Cycle 2/Day 1) in fed state patients (omitting the evening dose of PST3.1a on CID1 and C2D1).
In step 2, three DL (DL2a to 4a) with PST3.1a administered to fasted patients at the dose of 20, 40 and 70 mg/kg/day bid (i.e. twice a day), respectively, and/or three DL (DL2b to 4b) with PST3.1a administered to fed patients at the dose of 10, 20 and 40 mg/kg/day bid, respectively, could be opened.

For each patient included in the second step of the dose escalation, a full-course PK profiling is performed:
a. for DL2a, 3a and 4a, at C1D1 in fasted state;
b. for DL2b, 3b and 4b at C1D1 in fed state;
c. for DL2b only, 4 days before beginning treatment (C1D4), in fasted state.

### Expansion Phase

In the expansion phase, the safety profile and possible efficacy is further characterized in patients with GBM (23 patients, cohort 1), TNBC (15 patients, cohort 2) and other selected solid tumours selected by the PSC on the basis of preclinical pharmacological data and of the antitumour activity observed during the Dose Escalation Phase if any (30 patients, cohort 3) treated at the RP2D.

### Objectives and Endpoints

### Primary Objective and Endpoint:

To better characterize the safety profile of PhOx430 in three cohorts of patients affected by GBM, triple-negative breast cancer, and selected types of solid tumours respectively.

### Secondary Objectives and Endpoints:

To further characterize the pharmacokinetics (PK) profile of PhOx430 in GBM, TNBC and other selected types of solid tumour patients by establishing the PK profile of the drug.

To evaluate the efficacy of PhOx430 by determining the overall response rate (ORR), the progression-free survival (PFS) and the overall survival (OS).

### Treatment

### Drug Supply and Preparation

PST3.1a is supplied as a drinkable solution in vials of 2 mL and 10 mL. Patients are provided with an appropriate number of 2 mL and 10 mL vials, as well as with a specific dosing device, in order to combine them to reach the appropriate volume. Volumes of DP to be administered at each dosing occasion are rounded in a standardized way according to patient body weight range, as described in Table 2.

TABLE 2
Volumes of PST3.1a drug product (60 mg/mL) intended to be administered
to patients based on their weight and dose level

| | | Patient body weight range | | | | |
|---|---|---|---|---|---|---|
| Dose levels | Dose | 40-49 kg | 50-59 kg | 60-69 kg | 70-79 kg | 80-99 kg |
| (mg/kg/day) | (mg/kg/adm.) | Volume per administration | | | | |
| 5 | 2.5 | 2.0 | 2.5 | 3.0 | 3.0 | 3.5 |
| 10 | 5 | 4.0 | 4.5 | 5.5 | 6.5 | 7.0 |
| 20 | 10 | 7.5 | 9.0 | 11.0 | 13.0 | 14.0 |
| 40 | 20 | 15.0 | 18.0 | 22.0 | 25.0 | 28.0 |
| 70 | 35 | 26.0 | 32.0 | 38.0 | 44.0 | 50.0 |

All volumes are expressed in mL.

A maximum number of 5 vials (regardless of volume) has been set to be used by a patient on each dosing occasion.

### Dosing

PST3.1a is administered per os every 12±1 hours to fasted patients or after a regular meal, according to the schedule of the DL in which the patient is included. Fasting is defined as no food intake for 2 hours before and two hours after study drug administration. Cycles is of 21 days. If treatment is delayed for reasons other than toxicity, a maximal delay of 1 day is accepted.

### Results

The results obtained are summarized in Table 3 below.

TABLE 3

| | | | | | | | | Pre | Number of |
|---|---|---|---|---|---|---|---|---|---|
| | Fasted | | | AUC | T1/2 | C-max | T-Max | dosing | completed |
| DL | patients | Tumors | Tox | (h*ng/mL) | (h) | (ng/mL) | (h) | (ng/mL) | cycles |
| DL1 | 10001001 | Colorectal | No | 310 | 9.2 | 42 | 1.5 | 25.7 | 2 cycles |
| | | Cancer | DLT | | | | | | |
| | 15001001 | Pancreatic | No | 225 | 8.1 | 28 | 4 | 12.8 | 3 cycles |
| | | cancer | DLT | | | | | | |
| DL2 | 15001002 | GBM | No | 340 | 7.9 | 67.7 | 1 | 20.6 | 2 cycles |
| | | | DLT | | | | | | |
| DL3 | 15001005 | Uterine | No | 336.5 | N/A | 56.2 | 1.5 | 25.4 | 3 cycles |
| | | cancer | DLT | | | | | | |
| | 10001004 | Cholangio- | No | 650.6 | N/A | 97.6 | 2 | 46.3 | 2 cycles |
| | | carcinoma | DLT | | | | | | |
| | 10001005 | Colorectal | No | 626.6 | N/A | 108.9 | 2 | (upcoming | 1 cycle |
| | | cancer | DLT | | | | | data) | |

DL1 = (10 mg/kg/day) − DL2 = (20 mg/kg/day) − DL3 = (40 mg/kg/day)

Conclusion: The tested dose are in the exposure range.
The comparison between fed and fasted patients is summarized in Table 4:

TABLE 4

| | | | | | | | Number |
|---|---|---|---|---|---|---|---|
| DL1 ONLY | | | AUC | T1/2 | C-max | T-Max | of cycles |
| (10 mg/kg/day) | patients | Tumors | (h*ng/mL) | (h) | (ng/mL) | (h) | completed |
| FASTED | 10001001 | Colorectal | 310 | 9.2 | 42 | 1.5 | 2 cycles |
| | | Cancer | | | | | |
| | 15001001 | Pancreatic | 225 | 8.1 | 28 | 4 | 3 cycles |
| | | cancer | | | | | |
| FED | 10001001 | Colorectal | 587 | 17.5 | 43 | 0.8 | 2 cycles |
| | | Cancer | | | | | |
| | 15001001 | Pancreatic | 426 | 11.7 | 27.9 | 2 | 3 cycles |
| | | cancer | | | | | |

Conclusion: A significative fed effect is observed.

### LIST OF REFERENCES

Dennis, J. W., Nabi, I. R., & Demetriou, M. (2009). Metabolism, Cell Surface Organization, and Disease. Cell, 139(7), 1229-1241. https://doi.org/10.1016/j.cell.2009.12.008.Metabolism
Garrido-Castro, A. C., Lin, N. U., & Polyak, K. (2019). Insights into molecular classifications of triple-negative breast cancer: Improving patient selection for treatment. Cancer Discovery, 9(2), 176-198. https://doi.org/10.1158/2159-8290.CD-18-1177
Henley, S. J., Ward, E. M., Scott, S., Ma, J., Anderson, R. N., Firth, A. U., . . . Kohler, B. A. (2020). Annual report to the nation on the status of cancer, part I: National cancer statistics. Cancer, 126(10), 2225-2249. https://doi.org/10.1002/cncr.32802
Huang, M., O& #039; Shaughnessy, J., Zhao, J., Haiderali, A., Cortés, J., Ramsey, S. D., . . . Fasching, P. A. (2020). Association of Pathologic Complete Response with Long-Term Survival Outcomes in Triple-Negative Breast Cancer: A Meta-Analysis. Cancer Research, 80(24), 5427 LP—5434. https://doi.org/10.1158/0008-5472.CAN-20-1792
Kizuka, Y., & Taniguchi, N. (2016). Enzymes for N-Glycan branching and their genetic and nongenetic regulation in cancer. Biomolecules, 6(2), 1-21. https://doi.org/10.3390/biom6020025
Noch, E. K., Ramakrishna, R., & Magge, R. (2018). Challenges in the Treatment of Glioblastoma: Multisystem Mechanisms of Therapeutic Resistance. World Neurosurgery, 116, 505-517. https://doi.org/10.1016/j.wneu.2018.04.022
Patel, A. P., Fisher, J. L., Nichols, E., Abd-Allah, F., Abdela, J., Abdelalim, A., . . . Fitzmaurice, C. (2019). Global, regional, and national burden of brain and other CNS cancer, 1990-2016: a systematic analysis for the Global Burden of Disease Study 2016. The Lancet Neurology, 18(4), 376-393. https://doi.org/10.1016/S1474-4422(18)30468-X
Scott, D. A., Casadonte, R., Cardinali, B., Spruill, L., Mehta, A. S., Carli, F., . . . Drake, R. R. (2019). Increases in Tumor N-Glycan Polylactosamines Associated with Advanced HER2-Positive and Triple-Negative Breast Cancer Tissues. Proteomics. Clinical Applications, 13(1), e1800014. https://doi.org/10.1002/prca.201800014
Yamamoto, H., Swoger, J., Greene, S., Saito, T., Hurh, J., Sweeley, C., . . . Moskal, J. R. (2000). Beta1,6-N-acetylglucosamine-bearing N-glycans in human gliomas: implications for a role in regulating invasivity. Cancer Research, 60(1), 134-142

1. A method for treating cancer and/or reducing or preventing the appearance of metastases in a human patient afflicted with a cancer, the method comprising administering a compound of formula (1) to the human patient, wherein the compound of formula (1) is administered with a daily dose from 1 mg/kg to 80 mg/kg, and wherein the compound has the following formula (1): 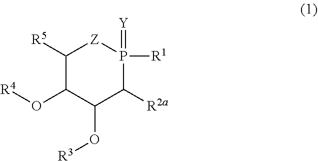
wherein Y represents an oxygen, a sulfur or a selenium atom,
Z represents O, S, Se, NH or a NR₆ group, wherein R₆ is an aryl or an optionally substituted alkyl group,
R¹ represents a hydrogen atom, an optionally substituted alkyl group or an aryl group,
R^{2a} represents a hydrogen atom, halogen, azide (N₃), a carbonate or dithiocarbonate group, a 1H-[1,2,3]triazolyl group or a group —X—R², wherein
X represents an oxygen, a sulfur, a selenium atom, a NH or NR₇ group, R₇ being an optionally substituted aryl or alkyl group; and;
R2 represents an aryl group, an optionally substituted alkyl group, a hydrogen atom, a trichloroacetimidate group (—C(═NH)CCl₃), an acyl, formyl, sulfonyl, sulfinyl, tert-butyldiphenylsilyl, allyl group, a saccharyl, ester, amide, thioamide, sulfonamide group, or X—R2 represents a P(O)R₂R₆ group, in which R₂ and R₆ represent independently from each other an aryl group, an optionally substituted alkyl group, OH, an alkoxy or an aryloxy group,
R³ and R⁴ represent independently from each other an aryl, an optionally substituted alkyl group, an hydrogen atom, a trichloroacetimidate group, an acyl, formyl, sulfonyl, sulfinyl, tert-butyldiphenylsilyl group, an allyl, ester, amide, thioamide, sulfonamide group, or R³ and
R⁴ taken together form a divalent radical of formula —R³—R⁴—,
R⁵ represents a hydrogen atom or a hydrocarbon group comprising one or more heteroatoms.

2. The method according to claim 1, wherein the compound is administered with a daily dose from 2 mg/kg to 70 mg/kg.

3. The method according to claim 1, wherein the cancer is selected from non-small-cell lung carcinoma (NSCLC), small-cell lung carcinoma (SCLC), breast cancer, oesophageal cancer, melanoma, gastric cancer, glioblastoma multiform, small bowel cancer, colorectal cancer, anal cancer, pancreatic cancer, ovarian cancer, uterine cancer, cervical cancer, hepatocarcinoma.

4. The method according to claim 1, wherein the compound is administered orally.

5. The method according to claim 1, wherein the compound is administered from 2 to 6 times per 24 hours.

6. The method according to claim 1, wherein the compound is administered from 2 or 3 times per 24 hours.

7. The method according to claim 1, wherein the compound is administered in a fasted or fed subject.

8. The method according to claim 1, wherein the compound is of formula (1) with Y═Z═O.

9. The method according to claim 1, wherein R⁵ is selected from the following groups: 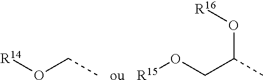
wherein R¹⁴, R¹⁵ and R¹⁶ represent, independently from each other, a hydrogen atom, an aryl group, an optionally substituted alkyl group, a trichloroacetimidate group, an acyl, formyl, sulfonyl, sulfinyl, tert-butyldiphenylsilyl, allyl, ester, amide or a sulfonamide group, or R¹⁵ and R¹⁶, taken together, form a divalent radical of formula —R¹⁵—R¹⁶—.

10. The method according to claim 1, wherein R¹ is a phenyl group and/or X—R2 is OH and/or R³ and R⁴ represent a benzyl group.

11. The method according to claim 1, wherein the compound of formula (1) is 3-Hydroxy-4,5-bis-benzyloxy-6-benzyloxymethyl-2-phenyl-2-oxo-2λ5-[1,2]oxaphosphinane.

12. The method according to claim 1, wherein the compound has the following Formula (I):

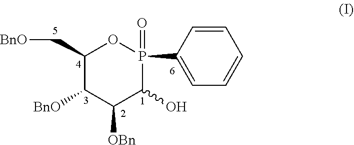 [SMILES: O=[P@@]1(c2ccccc2)O[C@H](COCc2ccccc2)[C@@H](OCc2ccccc2)[C@H](OCc2ccccc2)C1O]

13. The method according to claim 12, wherein the compound of Formula (I) is in a crystalline form characterized by powder x-ray diffraction reflections at about 8.65, 16.06, 16.53, 19.16 and 21.05±0.2 degrees two-theta.

14. The method according to claim 13, wherein the compound is further characterized by powder x-ray diffraction reflections at about 14.04, 17.69, 19.66, 22.02 and 25.12±0.2 degrees two-theta.

15. The method according to claim 13, wherein the compound is further characterized by powder x-ray diffraction pattern as depicted in FIG. 1.

16. The method of claim 1, wherein —R³—R⁴— represents an isopropylidene, benzylidene, diphenyl methylidene, cyclohexyl methylidene group, and their substituted analogues.

17. The method of claim 1, wherein the one or more heteroatoms are selected from oxygen, sulfur and nitrogen.

18. The method according to claim 2, wherein the compound is administered with a daily dose from 2 mg/kg to 60 mg/kg.

19. The method according to claim 5, wherein the compound is administered from 2 to 4 times per 24 hours.

20. The method of claim 9, wherein —R¹⁵—R¹⁶— represents an isopropylidene, benzylidene, diphenyl methylidene, or a cyclohexyl methylidene group, and their substituted analogues.
